(19) United States
(12) Patent Application Publication
(10) Pub. No.: US 2008/0138412 A1
(43) Pub. Date: Jun. 12, 2008

(54) Sustained release alfuzosin hydrochl formulation and method for their production

(76) Inventors: Fu-Yung Lin, Fongyuan City (TW); Fang-Hsiung Hsiao, Liu-Ying Hsiang (TW); Ya-Ching Chang Chien, Kaohsiung (TW)

Correspondence Address: Andrews & Kurth, L.L.P., Suite 1100, 1350 I Street N.W., Washington, DC, 20005, US

(21) Appl. No.: 11/634,072

(22) Filed: Dec. 6, 2006

(51) Int. Cl. A61K 9/22; A61K 31/517; A61K 9/00

(52) U.S. Cl. 424/468; 514/266.24; 424/400

(57) ABSTRACT

A sustained release alfuzosin hydrochloride formulation contains alfuzosin hydrochloride as about 1% to about 5% by weight of the formulation, hydrophilic polymers as about 35% to about 75% by weight of the formulation, hydrophobic polymers as about 10% to about 30% by weight of the formulation, disintegrating agents as 10% to 30% by weight of the formulation and a binder as about 2% to about 12% by weight of the formulation. The hydrophilic and hydrophobic polymers are used as the release-modulating agent to control the dissolution profile of the alfuzosin hydrochloride formulation so that the formulation releases alfuzosin hydrochloride slowly and continuously as the formulation passed through the gastrointestinal tract. The present invention also relates to a method for preparing the above formulation.

### BACKGROUND OF THE INVENTION

1. Field of Invention

The present invention relates to a sustained release alfuzosin hydrochloride formulation and a method for their productions.

2. Description of the Related Art

Alfuzosin hydrochloride is administered orally in the symptomatic treatment of benign prostatic hypertrophy. Alfuzosin hydrochloride has a short half-life of about 9 hours and displays a marked increase in the absorption at the duodenum-jejunum level. Now, two traditional oral forms are available in many countries. One is an immediate release tablet that contains 2.5 mg alfuzosin hydrochloride and is administered three times per day. The other is a sustained release tablet that contains 10 mg alfuzosin hydrochloride and is administered once a day. However, the immediate release tablet increases drug concentration in patients' plasma rapidly, and a high drug concentration shows a peak effect and terrible side effects after administering the immediate release tablet.

Most sustained release formulations provide a stable releasing rate to release an active substance, and the releasing rate is not affected by the pH value in the gastrointestinal tract. U.S. Pat. No. 6,149,940 and Taiwan Patent No. 522,023 describe a sustained release alfuzosin hydrochloride formulation that has three layers to control the releasing rate of alfuzosin hydrochloride. U.S. Pat. No. 5,589,190 describes a sustained release alfuzosin hydrochloride formulation that has a pH-dependent outer film-coat to control the releasing rate of alfuzosin hydrochloride. U.S. Pat. No. 5,589,190 also describes a sustained release alfuzosin hydrochloride formulation that has two different control release agents with two different releasing rates to control the release rate of alfuzosin hydrochloride. WO 2004/037,228 describes an alfuzosin hydrochloride formulation that contains bi-layers or multiple layers. The alflizosin hydrochloride formulation may contain a layer of the active substance of alfuzosin hydrochloride and one or more layers of different control releasing agents to control the release rate of alfuzosin hydrochloride.

Furthermore, WO 2004/037,228 discloses a formulation of a matrix controlled-release system, and the excipient in the formulation may be hydroxpropyl methylcellulose or hydroxypropyl cellulose. However, the compatibility of hydroxypropyl methylcellulose will be affected by kinds and concentrations of salts. Further, the precipitation temperature of hydroxypropyl methylcellulose in water is also affected by the concentration of sodium chloride or sugar. Therefore, such a matrix controlled-release system will cause more problems after oral administration in vivo.

In conclusion, the foregoing patents provide a stable release rate to release alfuzosin hydrochloride, but the methods described in the foregoing patents are complex and expensive. For example, U.S. Pat. No. 6,149,940 and Taiwan Pat. No. 522023 disclose a three layer formulation containing alfuzosin hydrochloride that requires a special tableting machine that is expensive and time consuming to operate. U.S. Pat. No. 5,589,190 and WO 2004/037,228 disclose a sustained release tablet that comprises one or multiple film-coats so the process of making the tablet must be controlled strictly to prevent undesirable conditions. The foregoing conventional procedures will increase cost, are time consuming and are not the best way to produce the sustained release alfuzosin hydrochloride formulation.

Therefore, a need still exists in the related art to provide a sustained release alfuzosin hydrochloride formulation that overcomes the complex production procedure and high cost problems, and maintains the desired sustained release effect.

### SUMMARY OF THE INVENTION

An objective of the present invention is to provide a sustained release alfuzosin hydrochloride formulation.

Another objective of the present invention is to provide a method for preparation of the sustained release alfuzosin hydrochloride formulation.

A sustained release alfuzosin hydrochloride formulation in accordance with the present invention contains about 1% to about 5% by weight of alfuzosin hydrochloride, about 35% to about 75% by weight of a hydrophilic polymer, about 10% to about 30% by weight of a hydrophobic polymer, about 2% to about 12% by weight of a binder and about 10% to about 30% by weight of a disintegrating agent. The hydrophilic polymer and the hydrophobic polymer are formed a release-modulating agent to control the dissolution profile of alfuzosin hydrochloride formulation so that the formulation releases alfuzosin hydrochloride slowly and continuously as the formulation passed through the gastrointestinal tract.

Other aspects, advantages and novel features of the invention will become more apparent from the following detailed description when taken in conjunction with the accompanying drawings.

### BRIEF DESCRIPTION OF THE DRAWINGS

FIG. 1 is a graph of the dissolution profiles of a conventional sustained release alfuzosin hydrochloride formulation and the sustained release alfuzosin hydrochloride formulation in Examples 1 and 2 of the present invention in a pH 4.5 phosphate buffer;

FIG. 2 is a graph of the dissolution profiles of the sustained release alfuzosin hydrochloride formulation in Examples 3, 4 and 5 of the present invention in a pH 4.5 phosphate buffer; and

FIG. 3 is a graph of the alfuzosin hydrochloride concentration in human plasma after oral administration of a conventional sustained release alfuzosin hydrochloride formulation (reference) or the sustained release alfuzosin hydrochloride formulation in Example 5 of the present invention.

### DETAILED DESCRIPTION OF THE INVENTION

The present invention comprises sustained release alfuzosin hydrochloride formulations and a method to prepare the sustained release alftizosin hydrochloride formulations.

The sustained release alfuzosin hydrochloride formulations comprise a therapeutically effective amount of alfuzosin hydrochloride, hydrophilic polymers, binders, hydrophobic polymers and disintegrating agents. The therapeutic effective amount of alfuzosin hydrochloride is the active principle, is small particles and is about 1% to about 5% by weight of the formulation depending on the desired dosage. The hydrophilic polymers are 35% to 75% by weight of the formulation. The hydrophilic polymer may be one or more than one of hydroxymethyl cellulose, hydroxyethyl cellulose, hydroxypropyl methylcellulose of MW 1,000 to 100,000, hydroxypropyl cellulose or carboxyrinyl polymer. The hydrophobic polymers and the hydrophilic polymers are release control agents. The hydrophobic polymers are mixed in proportion to the hydrophilic polymers to control release of the active principle and are about 10% to about 30% by weight of the formulation. The hydrophobic polymers may be one or more than one of ethyl cellulose, magnesium stearate, palmitate, hydrogenated oil, wax or mono-, di- or tri-substituted glyceride. The binders provide an adhesive force to hold substituents of the formulations together with high hardness and are about 2% to about 12% by weight of the formulation. The disintegrating agents are about 10% to about 30% by weight of the formulation. The disintegrating agent may be one or more than one of mannitol, lactose, starches, sorbitol, xylitol, microcrystalline cellulose or substances that promote water passing into the mixture. People skilled in the art will know how to choice suitable disintegrating agents for the sustained release alfuzosin hydrochloride formulation.

The method for preparing the sustained release alfuzosin hydrochloride formulations simply comprises steps of (a) providing components of a therapeutically effective amount of alfuzosin hydrochloride, hydrophilic polymers, binders, hydrophobic polymers and disintegrating agents, (b) shifting the components, (c) forming a premixture (1) by mixing the hydrophilic polymers and the hydrophobic polymers, (d) forming a premixture (2) by adding the therapeutically effective amount of alfuzosin hydrochloride into the premixture (1), (e) forming a mixture by adding the binder and the disintegrating agents into the premixture (2), and (f) compressing the mixture to form tablets.

In the step of providing components, the therapeutically effective amount of alfuzosin hydrochloride is the active principle and about 1% to about 5% by weight of the formulation depending on the desired dosage, the hydrophilic polymers are release control agents and about 35% to about 75% by weight of the formulation, the hydrophobic polymers are a release control agent and about 10% to about 30% by weight of the formulation and mixed in proportion to the hydrophilic polymers to form a porous premixture (1) to control release of the active principle, the binder is about 2% to about 12% by weight of the formulation and the disintegrating agents are about 10% to about 30% by weight of the formulation.

In the shifting step, the components are shifted to obtain small sized particles.

In the forming a premixture (2) step, small particles of alfuzosin hydrochloride are imbedded in the porous premixture (1) formed by the hydrophilic polymers and the hydrophobic polymers.

In the forming a mixture step, the binders and the disintegrating agents are added to the premixture (2) and mixed well to form the mixture.

In the compressing the mixture to form tablets step, each tablet compressed from the mixture is a matrix controlled-release system that will continue to release alfuzosin hydrochloride and may have a diameter of 8.1 mm and a hardness of 3 to 14 Kp. When the tablet is taken orally, the tablet swells by absorbing water and becomes gelatinous. Then, water molecules pass into the matrix controlled-release system that comprises hydrophilic polymers and hydrophobic polymers, and alfuzosin hydrochloride diffuses from the hydrophilic polymers and the hydrophobic polymers and is released into the digestive tract.

The examples illustrate the following advantages of the sustained release alfuzosin hydrochloride formulations of the present invention:

1. The sustained release alfuzosin hydrochloride formulation in accordance with the present invention simply has to be compressed to tablets. Therefore, the method for preparing the sustained release alfuzosin hydrochloride formulation is simple and cost less than conventional methods.

2. The sustained release alfuzosin hydrochloride formulations was found surprisingly to exhibit excellent prolonged ingredient-releasing efficacy. Patients can benefit from a reduction in the frequency of taking such formulations.

The term “sustained release” as used herein refers to formulation or dosage units of this invention that are slowly and continuously dissolved and absorbed in the gastrointestinal tract over a period of time.

The term “sustained release formulations or dosage units” as used herein refers to formulations that are slowly and continuously dissolved and absorbed in the gastrointestinal tract over a period of about twenty four hours or more. Preferred sustained release formulations are those exhibiting similar alfuzosin concentrations in human plasma with the reference drug suitable for once daily administration with one tablet per dose as described below.

The term “immediate release” (“IR”) refers to formulations or dosage units that rapidly dissolve in vitro and are intended to be completely dissolved and absorbed in the gastrointestinal tract. Conventionally, such formulations release at least 90% of the active ingredient within 30 minutes of administration.

The term “matrix controlled-release system” as used herein refers to a system of the present invention that is a combination of hydrophilic polymers and hydrophobic polymers to control the active principle to be released in a prolonged period of time.

Unless defined otherwise, all technical and scientific terms used herein have the same meaning as commonly understood by one of ordinary skill in the art to which this invention belongs. Also, all publications, patent applications, patents, and other references mentioned herein are incorporated by reference.

The invention is further described with reference to the following non-limiting examples.

### EXAMPLES

The following examples illustrate various aspects of the present invention but do not limit the claims in any manner whatsoever.

### Example 1

### Method for Preparing Sustained Release Alfuizosin Hydrochloride Formulation and Sustained Release Alfuzosin Hydrochloride Formulation (1) (for 1,000 Tablets)

The components to prepare tablets containing 10.0 mg alfuzosin hydrochloride follow.

| Components | Amount | |
|---|---|---|
| Ethyl cellulose (Commercial name: Ethocel) | 20 | g |
| Hydroxypropyl methylcellulose K4M | 15 | g |
| Hydrogenated castor oil | 21 | g |
| Povidone | 8.5 | g |
| Lactose | 50 | g |
| Colloidal silica | 2 | g |
| Alfuzosin hydrochloride | 10 | g |
| Hydroxypropyl methylcellulose K100M | 68.5 | g |
| Magnesium stearate | 5 | g |

### Procedure:

A sustained release alfuzosin hydrochloride formulation in accordance with the present invention is prepared as follows:

1. 10 g of alfuzosin hydrochloride, 2 g of colloidal silica and 50 g lactose were mixed well for 2 minutes and shifted through a Sieve No. 40 to obtain a first premixture.

2. 15 g of hydroxypropyl methylcellulose K4M, 20 g of ethyl cellulose and 21 g of hydrogenated castor oil were mixed well for 3 minutes and shifted through a Sieve No. 40 to obtain a second premixture.

3. The first premixture and the second premixture were mixed well for 3 minutes to obtain a first mixture.

4. 8.5 g of povidone was dissolved in 14 ml pure water and stirred slowly to completely dissolve this component. The dissolved povidone was added to the first mixture obtained from step 3 and was kneaded until a suitable consistency was achieved and shifted through a Sieve No. 24 to obtain a second mixture. Then, the second mixture was dried at 50° C. for 3 to 4 hours until water content was less than 2% of the second mixture.

5. 68.5 g hydroxypropyl methylcellulose K100M shifted through a Sieve No. 40 was added to the second mixture and mixed well for 2 minutes to obtain a third mixture.

6. 5 g of magnesium stearate shifted through a Sieve No. 60 was added to the third mixture and mixed for 1 minute.

7. The well mixed third mixture obtained from step 6 was put in a rotary type tableting machine to compress the mixture into tablets with diameters of 8.1 mm, hardness of 3 to 8 Kp and weight of 200 mg.

### Example 2

### Sustained Release Alfuzosin Hydrochloride Formulation (2) and Method for its Production (for 1,000 Tablets)

The components to prepare tablets containing 10.0 mg alfuzosin hydrochloride follow.

| Components | Amount |
|---|---|
| Ethyl cellulose (Commercial name: Ethocel) | 30 g |
| Hydroxypropyl methylcellulose K4M | 35 g |
| Hydrogenated castor oil | 21 g |
| povidone | 10 g |
| Microcrystalline cellulose | 28 g |
| Mannitol | 10 g |
| Colloidal silica | 4 g |
| Alfuzosin hydrochloride | 10 g |
| Hydroxypropyl methylcellulose K100M | 72 g |
| Magnesium stearate | 7 g |

### Procedure:

1. 10 g of alfuzosin hydrochloride, 4 g of colloidal silica and 10 g mannitol were mixed well for 2 minutes and shifted through a Sieve No. 40 to obtain a first premixture.

2. 35 g of hydroxypropyl methylcellulose K4M, 30 g of ethyl cellulose, 28 g microcrystalline cellulose and 21 g of hydrogenated castor oil were mixed well for 3 minutes and shifted through a Sieve No. 40 to obtain a second premixture.

3. The first premixture and the second premixture were mixed well for 3 minutes to obtain a first mixture.

4. 10 g of povidone was dissolved in 18 ml pure water and stirred slowly to completely dissolve this component. The dissolved povidone was added to the first mixture obtained from step 3 and was kneaded until a suitable consistency was achieved and then shifted through a Sieve No. 24 to obtain a second mixture. Then, the second mixture was dried at 50° C. for 3 to 4 hours until water content was less than 2% of the second mixture.

5. 72 g hydroxypropyl methylcellulose K100M shifted through a Sieve No. 40 was added to the second mixture and mixed well for 2 minutes to obtain a third mixture.

6. 7 g of magnesium stearate shifted through a Sieve No. 60 was added to the third mixture and mixed for 1 minute.

7. The well mixed third mixture obtained from step 6 was put in a rotary type tabletting machine to compress into tablets with diameters of 8.1 mm, hardness of 3 to 8 Kp and weight of 250 mg.

### Example 3

### Sustained Release Alfuzosin Hydrochloride Formulation (3) and Method for the Production Thereof (for 1,000 Tablets)

The components to prepare tablets containing 10.0 mg alfuzosin hydrochloride follow.

| Components | Amount | |
|---|---|---|
| Ethyl cellulose (Commercial name: Ethocel) | 25 | g |
| Hydroxypropyl methylcellulose K4M | 188 | g |
| Hydrogenated castor oil | 25 | g |
| povidone | 10 | g |
| Microcrystalline cellulose | 25 | g |
| Mannitol | 10 | g |
| Colloidal silica | 2 | g |
| Alfuzosin hydrochloride | 10 | g |
| Magnesium stearate | 5 | g |

### Procedures:

1. 10 g of alfuzosin hydrochloride, 2 g of colloidal silica and 10 g mannitol were mixed well for 2 minutes and shifted through a Sieve No. 40 to obtain a first premixture.

2. 128 g of hydroxypropyl methylcellulose K4M, 25 g of ethyl cellulose, 25 g microcrystalline cellulose and 25 g of hydrogenated castor oil were mixed well for 3 minutes and shifted through a Sieve No. 40 to obtain a second premixture.

3. The first premixture and the second premixture were mixed well for 3 minutes to obtain a first mixture.

4. 10 g of povidone was dissolved in 20 ml pure water and stirred slowly to completely dissolve this component. The dissolved povidone was added to the first mixture obtained from step 3 and was kneaded until a suitable consistency was achieved and then shifted through a Sieve No. 24 to obtain a second mixture. Then, the second mixture was dried at 50° C. for 3 to 4 hours until water content was less than 2% of the second mixture.

5. 60 g hydroxypropyl methylcellulose K4M shifted through Sieve No. 40 was added to the second mixture and mixed well for 2 minutes to obtain a third mixture.

6. 5 g of magnesium stearate shifted through a Sieve No. 60 was added to the third mixture and mixed for 1 minute.

7. The well mixed third mixture obtained from step 6 was put in a rotary type tableting machine to compress into tablets with diameters of 8.1 mm, hardness of 3 to 8 Kp and weight of 250 mg.

### Example 4

### Sustained Release Alfuzosin Hydrochloride Formulation (4) and Method for Their Production (for 1,000 Tablets)

The components to prepare tablets containing 10.0 mg alfuzosin hydrochloride follow.

| Components | Amount | |
|---|---|---|
| Ethyl cellulose (Commercial name: Ethocel) | 33 | g |
| Hydroxypropyl methylcellulose K4M | 230 | g |
| Hydrogenated castor oil | 25 | g |
| povidone | 10 | g |
| Microcrystalline cellulose | 25 | g |
| Mannitol | 10 | g |
| Colloidal silica | 1 | g |
| Alfuzosin hydrochloride | 10 | g |
| Magnesium stearate | 6 | g |

### Procedures:

1. 10 g of alfuzosin hydrochloride, 1 g of colloidal silica and 10 g mannitol were mixed well for 2 minutes and shifted through a Sieve No. 40 to obtain a first premixture.

2. 130 g of hydroxypropyl methylcellulose K4M, 10 g of ethyl cellulose, 25 g microcrystalline cellulose and 25 g of hydrogenated castor oil were mixed well for 3 minutes and shifted through a Sieve No. 40 to obtain a second premixture.

3. The first premixture and the second premixture were mixed well for 3 minutes to obtain a first mixture.

4. 10 g of povidone was dissolved in 36 ml pure water and stirred slowly to completely dissolve this component. The dissolved povidone was added to the first mixture obtained from step 3 and was kneaded until a suitable consistency was achieved and then shifted through a Sieve No. 24 to obtain a second mixture. Then, the second mixture was dried at 50° C. for 3 to 4 hours until water content was less than 2% of the second mixture.

5. 100 g hydroxypropyl methylcellulose K4M and 23 g ethyl cellulose shifted through a Sieve No. 40 were added to the second mixture and mixed well for 2 minutes to obtain a third mixture.

6. 5 g of magnesium stearate shifted through a Sieve No. 60 was added to the third mixture and mixed for 1 minute.

7. The well mixed third mixture obtained from step 6 was put in a rotary type tableting machine to compress into tablets with diameters of 8.1 mm, hardness of 3 to 8 Kp and weight of 350 mg.

### Example 5

### Sustained Release Alfuzosin Hydrochloride Formulation (5) and Method for the Production Thereof (for 1,000 Tablets)

The components to prepare a tablet containing 10.0 mg alfuzosin hydrochloride follow.

| Components | Amount | |
|---|---|---|
| Ethyl cellulose (Commercial name: Ethocel) | 32 | g |
| Hydroxypropyl methylcellulose K4M | 210 | g |
| Hydrogenated castor oil | 25 | g |
| Povidone | 10 | g |
| Microcrystalline cellulose | 45 | g |
| Mannitol | 10 | g |
| Colloidal silica | 1.5 | g |
| Alfuzosin hydrochloride | 10 | g |
| Magnesium stearate | 6.5 | g |

### Procedures:

1. 10 g of alfuzosin hydrochloride, 1.5 g of colloidal silica and 10 g mannitol were mixed well for 2 minutes and shifted through a Sieve No. 40 to obtain a first premixture.

2. 45 g of microcrystalline cellulose, 10 g of povidone and 25 g of hydrogenated castor oil were mixed well for 3 minutes and shifted through a Sieve No. 40 to obtain a second premixture.

3. 210 g hydroxypropyl methylcellulose K4M and 32 g ethyl cellulose were mixed for 2 minutes and shifted through a Sieve No. 40 to obtain a third premixture.

4. The first premixture, second premixture and third pre-mixture were mixed well for 3 minutes to obtain a mixture.

5. 6.5 g of magnesium stearate shifted through a Sieve No. 60 was added to the mixture and mixed for 1 minute.

6. The well mixed mixture obtained from step 5 was put in a rotary type tableting machine to compress into tablets with diameters of 8.1 mm, hardness of 8 to 14 Kp and weight of 350 mg.

### Example 6

### Dissolution Test

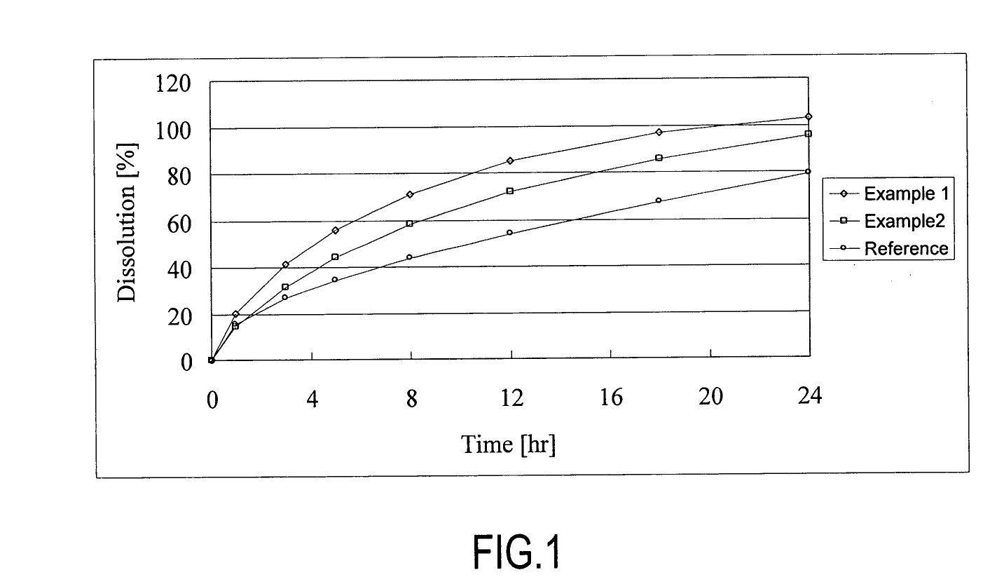

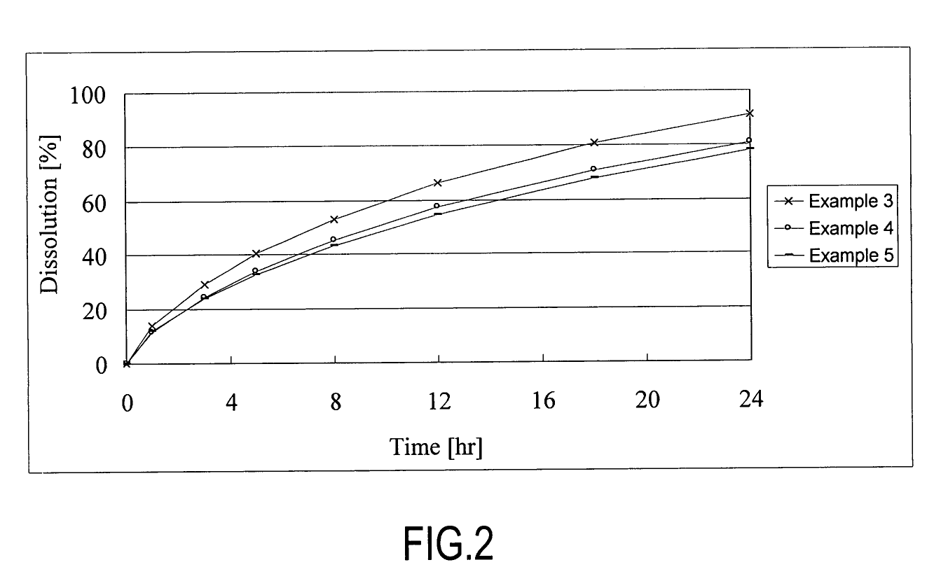

The tablets obtained from Examples 1 to 5 were compared with a conventional sustained release alfuzosin hydrochloride formulation (reference). The dissolution test was carried out under the instructions of the United States Pharmacopoeia (U.S.P.) 28^{th} edition. 900 ml of pH 4.5 phosphate buffer at 37.5°±0.5° C. was used as a dissolution solution. Tablets were paddled in a mixer at 50 rpm to test the dissolution rate. The results are shown in Table 1 and FIGS. 1 and 2.

As shown in Table 1 and FIGS. 1 and 2, the tablets obtained from Examples 1 to 5 had a stable releasing rate compared with the conventional sustained release alfuzosin hydrochloride formulation (reference) under the pH 4.5 phosphate buffer. The dissolution percentage of alfuzosin hydrochloride released from the tablets obtained from Examples 1 to 5 are higher than 75% with reference of the equal amount of the pure alfuzosin hydrochloride.

The dissolution rate of alfuzosin hydrochloride from tablets obtained from Examples 1 and 2 compared with an equal amount of conventional sustained release alfuzosin hydrochloride formulation (reference) under pH 4.5 phosphate buffer are shown in FIG. 1.

The dissolution percentage of alfuzosin hydrochloride released from the tablets, obtained from Examples 3 to 5 under pH 4.5 phosphate buffer are shown in FIG. 2.

TABLE 1
The dissolution rate of tablets obtained from Examples 1 to 5
during 24 hours

| Dissolution | | | | | | |
|---|---|---|---|---|---|---|
| percentage of | | | | | | |
| Alfuzosin | | | | | | |
| hydrochloride | | | | | | |
| released | | | | | | |
| [%]/hr | Ex 1 | Ex 2 | Ex 3 | Ex 4 | Ex 5 | Ref |
| 0.0 hr | 0.0 | 0.0 | 0.0 | 0.0 | 0.0 | 0.0 |
| 1.0 hr | 20.2 | 14.8 | 14.2 | 11.8 | 12.0 | 15.7 |
| 3.0 hr | 41.3 | 31.7 | 29.4 | 24.7 | 24.3 | 26.9 |
| 5.0 hr | 55.8 | 44.3 | 40.4 | 34.2 | 32.9 | 34.6 |
| 8.0 hr | 71.1 | 58.5 | 53.2 | 45.5 | 43.5 | 43.8 |
| 12.0 hr | 85.0 | 72.2 | 66.1 | 57.3 | 54.8 | 54.1 |
| 18.0 hr | 97.0 | 85.8 | 80.9 | 70.8 | 68.0 | 67.1 |
| 24.0 hr | 103.1 | 95.6 | 91.4 | 80.9 | 78.1 | 78.9 |

### Example 7

### Bioequivalence Study

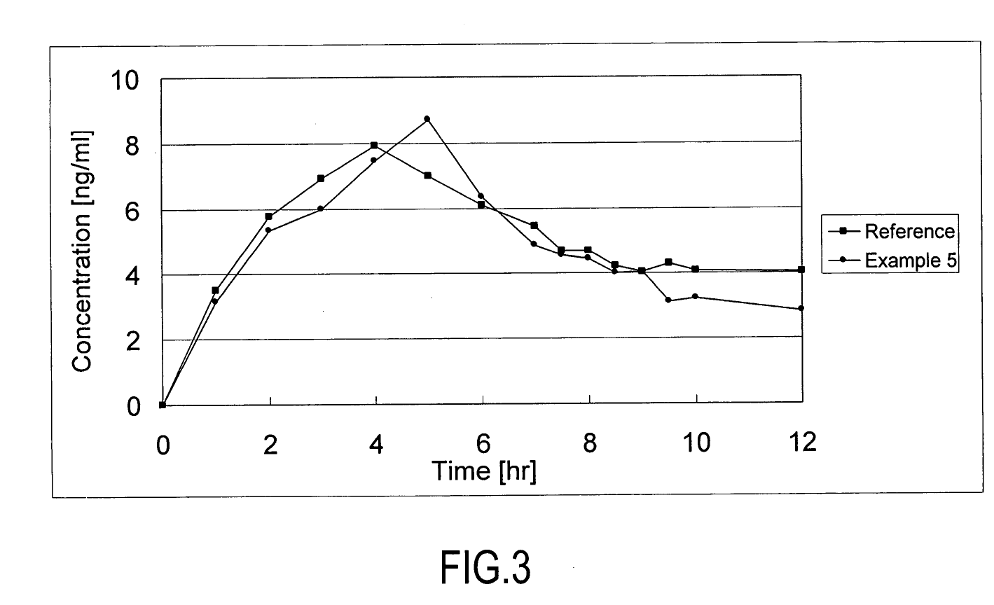

In the study of bioequivalence, tablets obtained from Example 5 and the conventional sustained release alfuzosin hydrochloride formulation (reference) were used and the concentration of alfuzosin hydrochloride in human plasma were measured. The alfuzosin hydrochloride concentration in human plasma after oral administration of the tablet containing 10.0 mg alftizosin hydrochloride, is shown in Table 2 and FIG. 3. With reference of FIG. 3, results show that tablets obtained from Example 5 and the conventional sustained release alfuzosin hydrochloride formulation have similar alfuzosin hydrochloride concentrations in human plasma and the bioavailability between the tablets obtained from Example 5 and the reference are both more than 12 hours.

TABLE 2
The concentration of alfuzosin hydrochloride in human
plasma [g/ml]

Alfuzosin conc.

| | | Tablets obtained |
|---|---|---|
| Time [hrs] | Reference | from Example 5 |
| 0 | 0.00 | 0.00 |
| 1 | 3.51 | 3.14 |
| 2 | 5.79 | 5.35 |
| 3 | 6.91 | 5.99 |
| 4 | 7.92 | 7.46 |
| 5 | 6.98 | 8.70 |
| 6 | 6.10 | 6.35 |
| 7 | 5.44 | 4.89 |
| 8 | 4.69 | 4.46 |
| 9 | 4.07 | 4.05 |
| 10 | 4.10 | 3.23 |
| 12 | 4.05 | 2.83 |

Various modifications and variations of the present invention will be recognized by those persons skilled in the art without departing from the scope and spirit of the invention. Although the invention has been described in connection with specific preferred embodiments, the invention as claimed should not be unduly limited to such specific embodiments. Indeed, various modifications of the described modes for carrying out the invention, which are obvious to those skilled in the art, are intended to be within the scope of the following claims.

What is claimed is:

1. A sustained release alfuzosin hydrochloride formulation comprising
alfuzosin hydrochloride being the active principle, being small particles and being about 1% to about 5% by weight of the formulation;
hydrophilic polymer(s) being about 35% to about 75% by weight of the formulation;
hydrophobic polymer(s) being about 10% to about 30% by weight of the formulation;
disintegrating agent(s) being about 10% to about 30% by weight of the formulation; and
binder(s) being about 2% to about 12% by weight of the formulation.

2. The formulation as claimed in claim 1, wherein the hydrophilic polymer(s) is/are hydroxymethyl cellulose, hydroxyethyl cellulose, hydroxypropyl methylcellulose of MW 1,000 to 100,000, hydroxypropyl cellulose or carboxyrinyl polymer.

3. The formulation as claimed in claim 1, wherein the disintegrating agent(s) is/are mannitol, lactose, starches, sorbitol, xylitol, microcrystalline cellulose or substances that promotes water passing into the mixture.

4. The formulation as claimed in claim 1, wherein the hydrophobic polymer(s) is/are ethyl cellulose, magnesium stearate, palmitate, hydrogenated oil, wax or mono-, di- or tri-substituted glyceride.

5. A method for preparing a sustained release alfuzosin hydrochloride formulation, comprising
providing components of
a therapeutically effective amount of alfuzosin hydrochloride as the active principle and about 1% to about 5% by weight of the formulation;
hydrophilic polymer(s) being about 35% to about 75% by weight of the formulation;
hydrophobic polymer(s) being about 10% to about 30% by weight of the formulation;
binder(s) being about 2% to about 12% by weight of the formulation; and
disintegrating agent(s) being about 10% to about 30% by weight of the formulation;
forming a premixture (1) by mixing the hydrophilic polymer(s) and the hydrophobic polymer(s);
forming a premixture (2) by adding the therapeutically effective amount of alfuzosin hydrochloride;
forming a mixture by adding the binder(s) and the disintegrating agent(s) to the premixture (2) and mixing well to form the mixture; and
compressing the mixture into tablets.

6. The method as claimed in claim 5, wherein the hydrophilic polymer(s) is/are hydroxymethyl cellulose, hydroxyethyl cellulose, hydroxypropyl methylcellulose of MW 1,000 to 100,000, hydroxypropyl cellulose or carboxyrinyl polymer.

7. The method as claimed in claim 5, wherein the disintegrating agent(s) is/are mannitol, lactose, starches, sorbitol, xylitol, microcrystalline cellulose or substances that promotes water passing into the mixture.

8. The method as claimed in claim 5, wherein the hydrophobic polymer(s) is/are ethyl cellulose, magnesium stearate, palmitate, hydrogenated oil, wax or mono-, di- or tri-substituted glyceride.

9. The method as claimed in claim 5, wherein hydrophilic polymer(s), hydrophobic polymer(s), alfuzosin hydrochloride, disintegrating agent(s) and binder(s) are shifting before mixing.

10. The method as claimed in claim 5, wherein the disintegrating agent(s) is/are mannitol, lactose, starches, sorbitol, xylitol, microcrystalline cellulose or substances that promotes water passing into the mixture.
